(19) United States
(12) Patent Application Publication
(10) Pub. No.: US 2011/0313345 A1
(43) Pub. Date: Dec. 22, 2011

(54) ULTRASONIC DEVICE FOR HARVESTING ADIPOSE TISSUE

(75) Inventor: Mark E. Schafer, Lower Gwynedd, PA (US)

(73) Assignee: Sound Surgical Technologies LLC, Louisville, CO (US)

(21) Appl. No.: 13/113,811

(22) Filed: May 23, 2011

Related U.S. Application Data
(60) Provisional application No. 61/347,378, filed on May 21, 2010.
(60) Provisional application No. 61/411,423, filed on Nov. 8, 2010.

(51) Int. Cl. A61M 1/00

(52) U.S. Cl. 604/22

(57) ABSTRACT

Described are embodiments of methods and devices for removing adipose tissue from a surgical site or location in a patient's body. The embodiments include a device that is used for infiltration, ultrasound, and aspiration of the surgical site. The device includes a cannula which serves to provide infiltration, conduct ultrasonic energy from an ultrasound generating device, and also provide a conduit for aspiration to a fluid system used for infiltration and collection of fluids. Embodiments provide for a fixed amount of infiltration fluid to be injected into the surgical site in specific ratio with the amount of lipoaspirate to be removed. The fixed amount of ultrasonic energy, both in amplitude and time, is delivered to the surgical site commensurate with the amount of infiltration and aspiration. The device also includes, in embodiments, a guide that limits the depth to which the cannula can be inserted into a patient.

### I. CROSS-REFERENCE TO RELATED APPLICATIONS

This application claims priority to U.S. Provisional Patent Application Ser. No. 61/347,378 filed May 21, 2010 entitled, “ULTRASONIC DEVICE DEDICATED TO HARVESTING OF ADIPOCYTES” and to U.S. Provisional Patent Application Ser. No. 61/411,423, filed Nov. 8, 2010 entitled, “ULTRASONIC DEVICE FOR HARVESTING ADIPOCYTES,” both provisional applications are hereby incorporated by reference in their entirety as if set forth herein in full.

### II. FIELD OF THE INVENTION

This invention relates to the field of ultrasonics and the application of ultrasonic energy in medicine, particularly as it relates to the “harvesting” of adipose tissue from an animal, e.g., a human patient, through the application of ultrasonic energy and aspiration of the disassociated adipose tissue from the patient.

### III. BACKGROUND OF THE INVENTION

In the past few years, the use of lipoaspirate material, that is, the material removed from a patient during liposuction, has expanded. Traditionally, the goal of the liposuction treatment was the removal of adipose tissue, including the “fat cells” (adipocytes) in the adipose tissue, in order to achieve some level of body contouring, and the lipoaspirate, composed of adipose tissue and tumescent fluid, was disposed of as a biological waste material.

Now, however, there is another distinct use for the lipoaspirate. It has been found that in addition to mature adipocytes, the lipoaspirate also contains pre-adipocytes (also known as Adipose Derived Stem Cells or “ADSCs”) and other cellular material such as leukocytes, neutrophils, etc. (the other cellular material being cumulatively referred to as the Stromal Vascular Fraction or “SVF”). The lipoaspirate can be separated into components, including “ADSCs.” The ADSCs can be processed to have a therapeutic purpose within the body, since, like other induced pluripotent stem cells, they can be used to regenerate nearly any other type of cell. See, for example, U.S. patent application entitled “Selective Lysing of Cells using Ultrasound,” Ser. No. 12/941,868 filed on Nov. 8, 2010, which is incorporated by reference herein. This is an area of great interest within the regenerative medicine community.

Typically, ADSCs are harvested during a cosmetic medical procedure employing liposuction, and then used primarily as an adjunct to a re-implantation of the patient's fat into his/her body. For example, fat may be extracted from the hips or stomach region and re-injected into the buttocks, breast, or face for an improved cosmetic appearance. The ADSCs are used to improve the percentage of injected material which remains viable and thrives in its new location within the body. Thus, currently, the aesthetic needs of the patient drives the overall procedure.

Soon, however, the therapeutic benefits of ADSC treatment will be shown to be sufficient to justify lipoaspiration independent of any cosmetic or aesthetic need of the patient. For example, a heart attack victim may be treated with ADSCs to stimulate the regeneration of heart muscle. In this case, the removal of fat will not be driven by cosmetic or aesthetic reasons.

Nevertheless, the current approach to fat removal is still grounded in the cosmetic and plastic surgery markets, in that the equipment and personnel operating the equipment are all of a sophistication and skill level commensurate with an aesthetic result. In other words, only plastic surgeons, dermatologists or other medical specialists with specific training in cosmetic procedures are able to operate the multifaceted ultrasonic equipment. The equipment is designed to facilitate the removal of cosmetically significant volumes of fat, in a way that provides the operator with the ability to “sculpt” the body for the desired (i.e., cosmetic) goal. The surgeon is therefore in control of infiltration amounts, ultrasound amounts, and aspiration.

What is needed is a system designed specifically to remove a small volume of lipoaspirate, suitable for processing into ADSCs, in a way that would be easily operated by clinical personnel who do not have specific training in cosmetic procedures. This would eliminate the need for plastic surgeons, etc. to be involved in therapeutic procedures (which may eventually occur in the Emergency Room or Interventional Radiology setting, at odd hours).

It has been shown that ultrasonic energy aids in the removal of fat through the action of cavitation and acoustic streaming. Ultrasound assisted liposuction equipment, specifically the VASER® from Sound Surgical Technologies (Louisville, Colo.), has been found to be uniquely suited for ADSC harvesting. It produces a smooth aspirate with small cell packets, with high cell viability. Also, it is more selective for harvesting adipose tissue as opposed to muscle, nerve or other cells.

Previous efforts have simply used the existing equipment for ADSC harvesting, either through manual means (Suction Assisted Liposuction, SAL) or using Ultrasound (UAL), or Water Jet (WAL, commercially available as the BodyJet system). The prior solutions comprise various devices and components effecting separate process steps which make for a difficult and cumbersome approach when the objective is to harvest ADSCs with minimal necrosis. Even when the VASER® is used for tissue fragmentation followed by aspiration of the fragmented tissue via the VentX® system (both available from Sound Surgical Technologies LLC of Louisville, Colo.), the optimum result is not achieved. The VASER® and VentX® systems are designed for a wide range of possible cosmetic potentials, with a wide range of output settings, multiple cannulae, an infiltration system capable of large dosages, an aspiration system concomitantly designed to handle liters of lipoaspirate. Even with proper instructions, it would require operation by a trained surgeon to produce the desired result. And the result may not be easily replicated on a repeated basis with confidence in the results for further processing of the harvested cells. In addition, the expense of the VASER® and VentX® prevents its widespread adoption to this focused, non-cosmetic application.

Even where fragmentation and aspiration are incorporated in a single device such as the Lysonix® system available from Mentor Corporation, Santa Barbara, Calif. there are significant issues with multiple components and process variables, expense and a high level of skill required for successful, reliable recovery of ASCSs for successful post processing into therapeutic stem cells. Of particular note, the Lysonix® system has been designed to operate at ultrasound frequencies which are not as appropriate for cell survival.

Thus, there is a need for an inexpensive, integrated system that can be operated by one of lesser medical skill than a surgeon to reliably extract ADSCs from a patient with minimal cell necrosis for the purpose of subsequently processing the cells into therapeutic stem cells for re-insertion into the same patient. The system should be designed to minimize the trauma that can be caused to a patient, and remove only the necessary amount of tissue from a patient. Such a system can be operated effectively with minimal removal of lipoaspirate, e.g., about 250 cc of lipoaspirate (exclusive of the injected infiltration material, which is also partially removed during the procedure).

### IV. BRIEF DESCRIPTION OF THE INVENTION

Embodiments of the present invention are directed to extracting lipoaspirate with ADSCs for regenerative medicine. The embodiments of the present invention are therefore different from systems and methods used in conventional liposuction procedures used for cosmetic purposes.

The present invention includes, in embodiments, therapeutic systems and methods that are self contained, and separate from any cosmetic liposuction equipment and methods. Systems embodying the invention combine the lipoaspiration processes of infiltration, energy deposition, and aspiration. Further, since the current standard approach to processing lipoaspirate down to ADSCs components uses a 250 cc container, embodiments provide for extracting this amount. This puts an upper bound on the amount of infiltration fluid (which is in proportion to the amount expected to be withdrawn), as well as the size of the container(s) used during the aspiration portion of the procedure.

The current invention includes the use of a cannula for infiltration, ultrasound exposure, and aspiration. Embodiments also provide for using a fixed amount of infiltration fluid, in specific ratio with the amount of lipoaspirate to be removed. The amount of ultrasonic energy is a fixed amount of ultrasonic energy, both in amplitude and time, commensurate with the amount of infiltration and aspiration is applied. The ultrasonic cannula used in embodiments of the present invention can also include holes that are positioned so as not to interfere with the ultrasonic action. The cannula is also designed to withstand the stresses of ultrasonic vibrations and deliver infiltration of fluid to an anatomical site and remove lipoaspirate, which includes adipocytes. Embodiment also provide for limiting the extent to which the cannula can be positioned within the patient, enhancing safety while naturally limiting the cosmetic utility of the device.

### V. BRIEF DESCRIPTION OF THE DRAWINGS

FIG. 1 is a schematic representation of an embodiment of a system for delivering infiltration fluid to a surgical site and removing lipoaspirate from the surgical site.

FIG. 2 is a schematic representation of a second embodiment of a system showing a different system for delivering infiltration fluid to a surgical site and removing lipoaspirate from the surgical site.

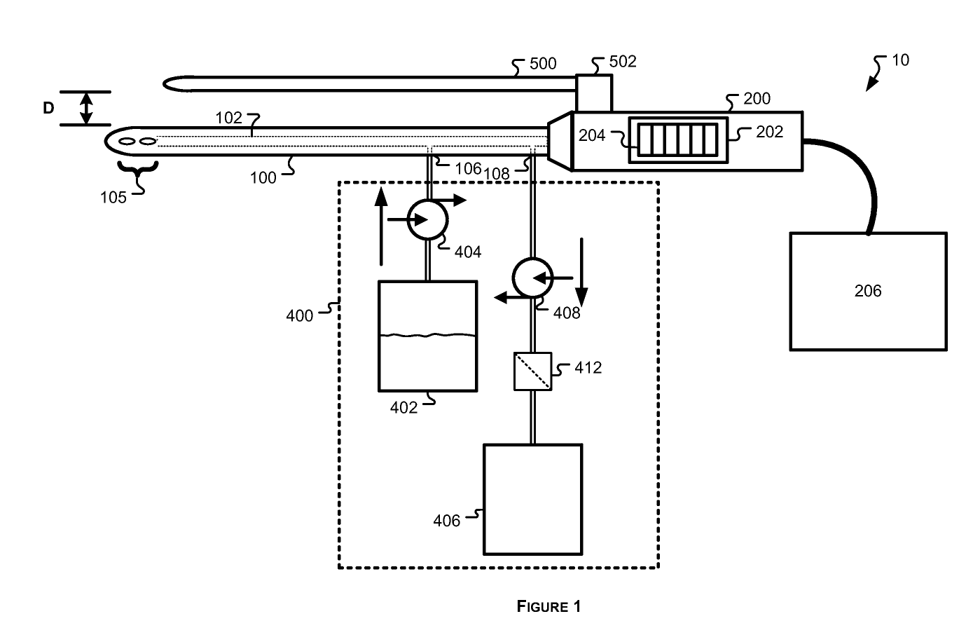

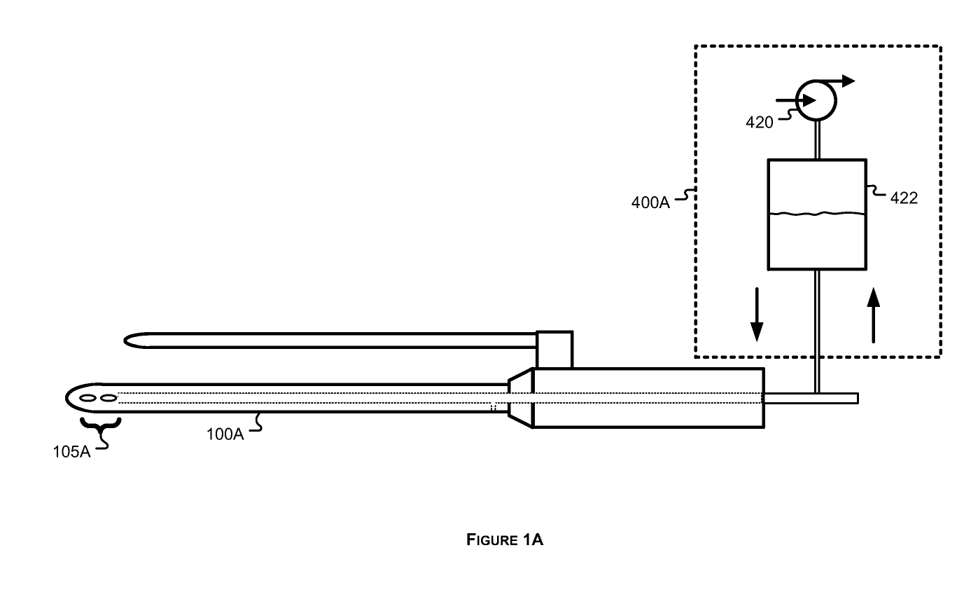

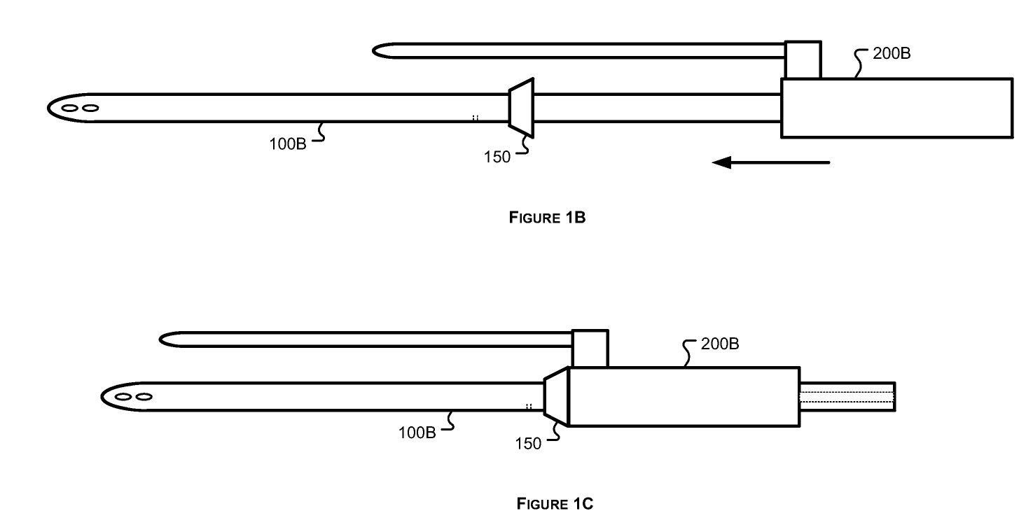

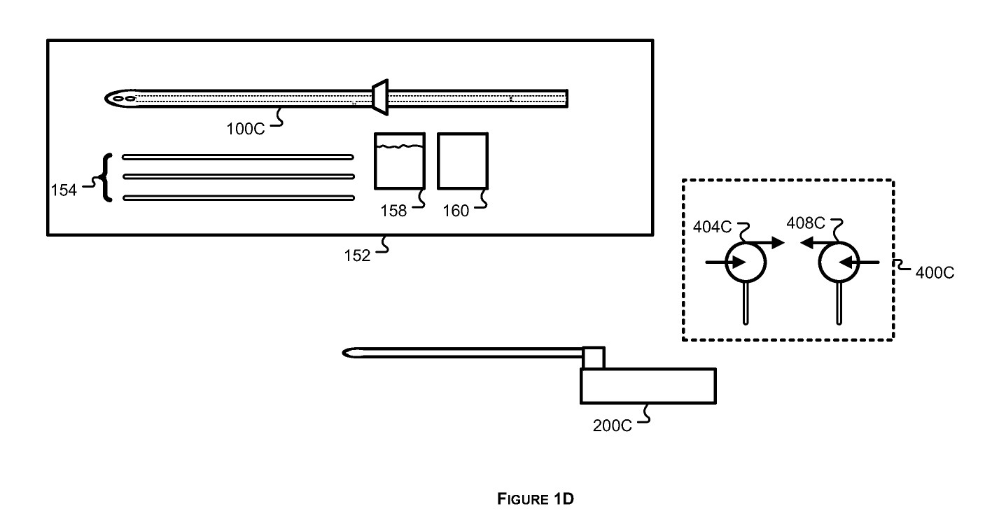

FIG. 1B illustrates an embodiment of a cannula that is removable from a handpiece that may be used in some embodiments.

FIG. 1C illustrates the cannula and handpiece of FIG. 1B where the handpiece is engaged with a stop on the cannula.

FIG. 1D is a schematic representation of an embodiment of a system that includes a kit of disposable components as well as components that are reused.

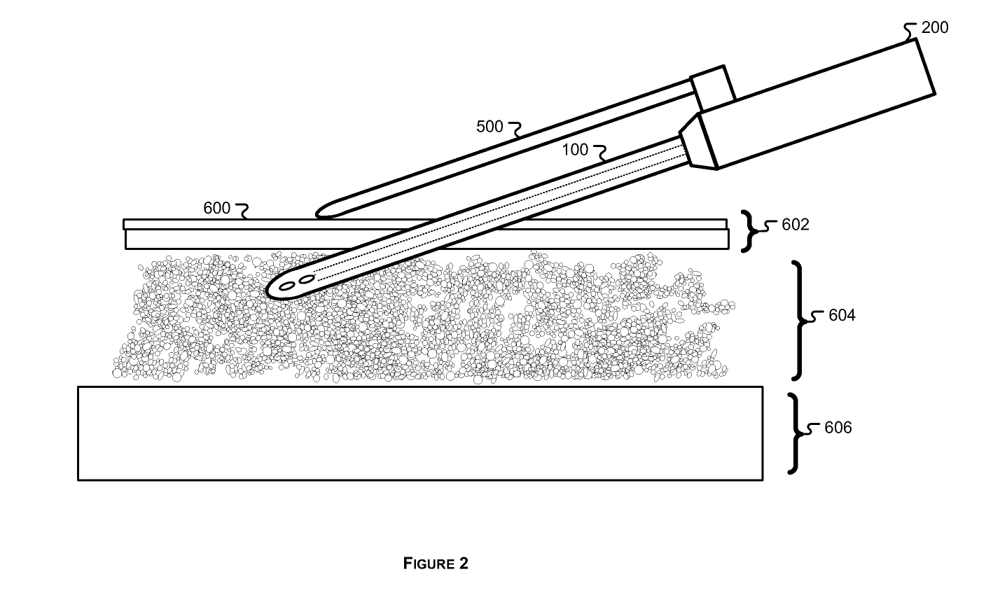

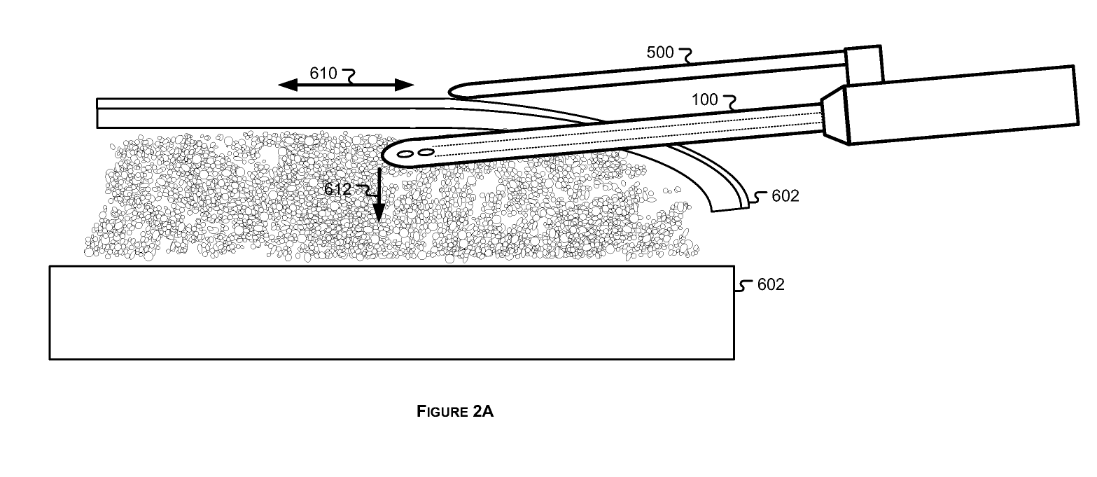

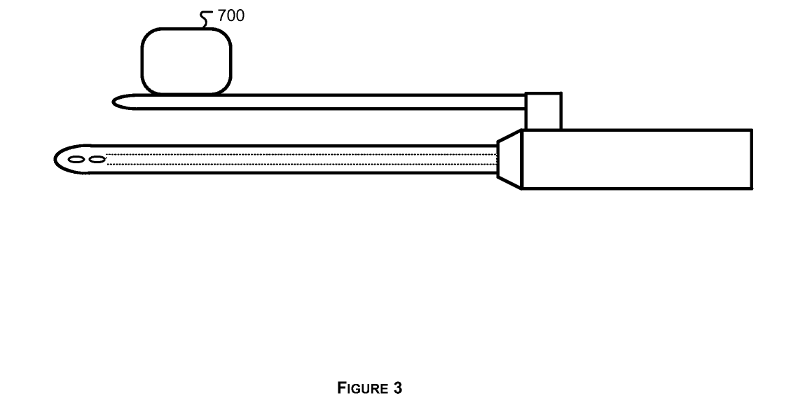

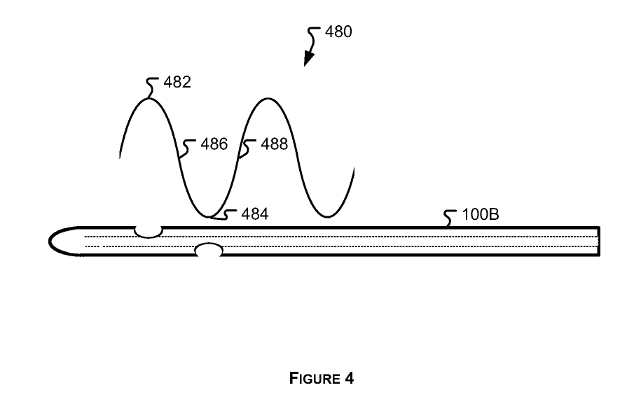

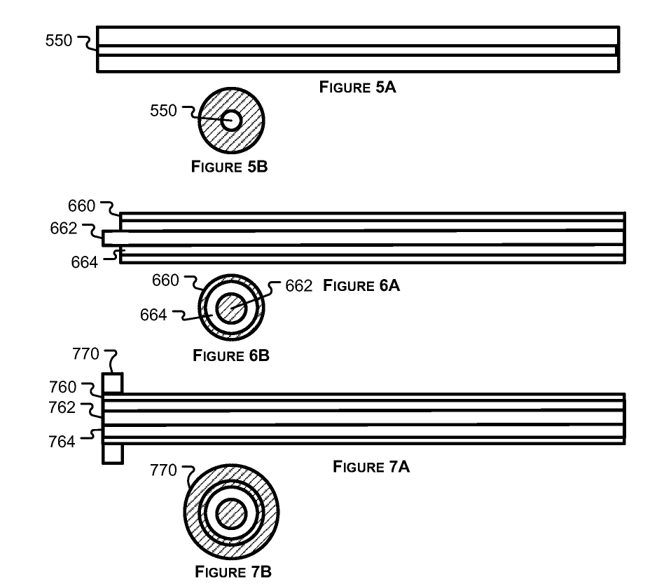

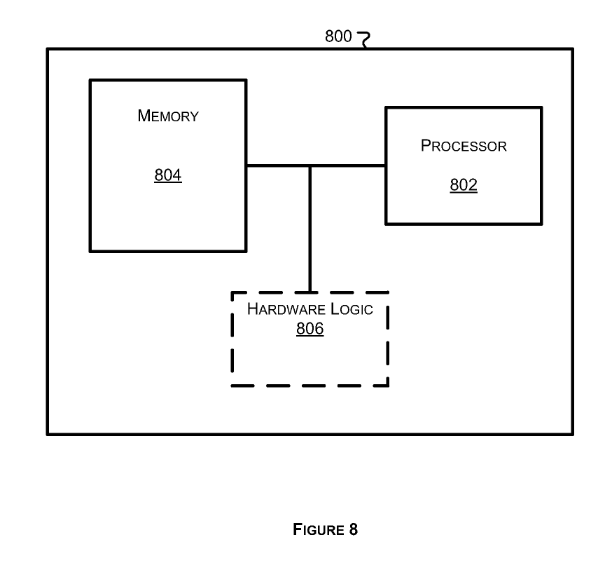

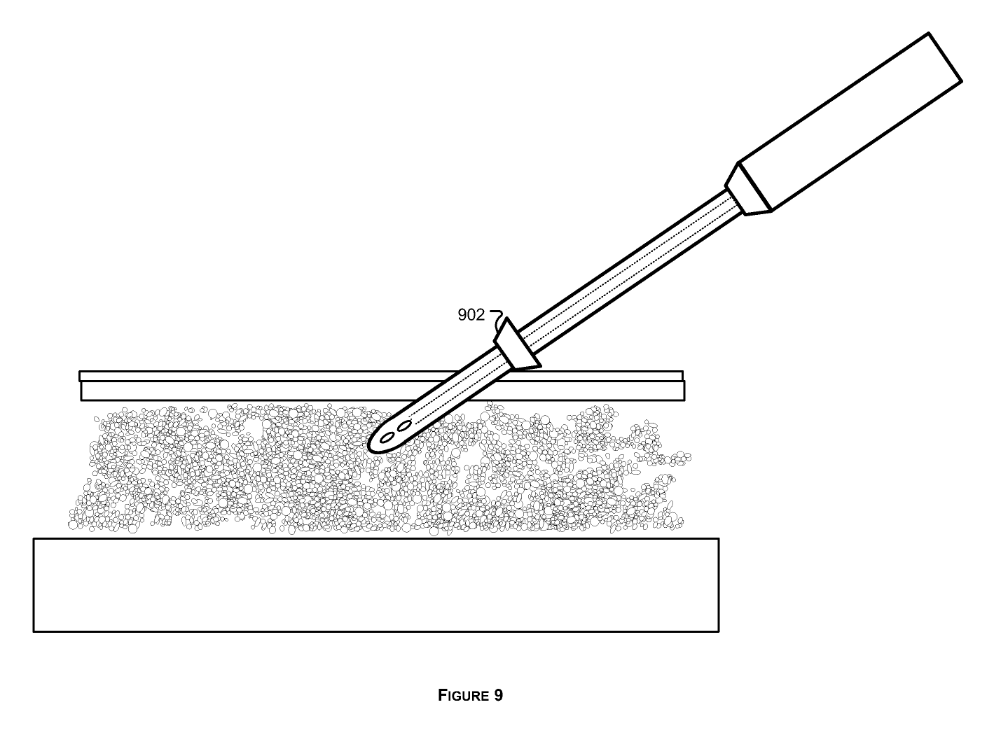

FIG. 2 illustrates an embodiment showing a cannula inserted into a patient through an incision in the skin and a guide that limits the depth to which the cannula can be inserted into the patient.

FIG. 2A illustrates the movement of the cannula, shown in FIG. 2, when inserted into a patient.

FIG. 3 shows an embodiment of a cannula with an ultrasonic imaging probe attached to the guide, allowing for imaging of the tissue beneath the skin.

FIG. 4 shows a cannula design with holes located at nodal locations of a wave transmitted by the cannula.

FIGS. 5A-7B show side and front views of different cannula designs that may be used with embodiments of the present invention.

FIG. 8 shows a block diagram of a computing device that can be used to control various features of the embodiments of the present invention.

FIG. 9 illustrates another embodiment showing a different guide that limits the depth to which a cannula can be inserted into a patient.

### VI. DETAILED DESCRIPTION OF THE INVENTION AND CERTAIN EMBODIMENTS

Embodiments of the present inventions include a method for the ultrasonic harvesting of adipose tissue from a medical patient utilizing a medical device for that purpose. The method includes providing an ultrasonic device capable of delivering ultrasonic energy to a surgical site in a patient and providing a cannula to be used for the application of infiltration fluid to the surgical site. The method includes application of ultrasonic energy to the surgical site, and aspiration of adipose tissue from the surgical site. The cannula in embodiments includes at least one hole for delivery of the infiltration fluid and for removal of the lipoaspirate. The hole is positioned so as not to interfere with the delivery of ultrasonic energy. The method further provides for delivering a fixed amount of infiltration fluid to the surgical site, in specific ratio with the amount of lipoaspirate to be removed and delivering a fixed amount of ultrasonic energy, both in amplitude and time, commensurate with the amount of infiltration and aspiration. Finally, the method involves removing the lipoaspirate using the cannula.

Some embodiments of the present invention have as a main, intended purpose the harvesting/removal of adipose tissue and adipose derived stem cells (ADSCs) in the adipose tissue for purposes other than any cosmetic benefits achieved by that removal. Those embodiments include devices dedicated and/or optimized for the harvesting of adipose tissue and ADSCs. This permits embodiments, including devices and methods, to be presented that can be optimized to achieve this result, can be utilized by someone who is skilled but not at the same level as a cosmetic, i.e., “plastic,” surgeon, and therefore can be manufactured and employed less expensively than a full, multipurpose UAL device designed for and used in cosmetic surgery. As described in greater detail below, the device is optimized for removal of a predetermined amount of lipoaspirate containing sufficiently viable tissue suitable for post-processing and/or re-injection by delivering a preset amount of infiltration fluid to a surgical site, applying a predetermined amount of ultrasonic energy to the surgical site, and removing the predetermined amount of lipoaspirate using a single cannula. Additionally, the device includes features that limit the depth to which the cannula can be inserted into a patient. These features allow the removal of adipose tissue and ADSCs safely and efficiently even by a user with less medical skill than typical of cosmetic surgeons.

Shown in FIG. 1 is an embodiment of a device 10 that is useful for removing adipose tissue from a surgical site or location, i.e., the location in a patient's body from which the adipose tissue will be harvested. The location of the surgical site may be, for example, the abdominal area which may be accessed through the umbilicus. In other instances, the surgical site may be the lower back region or the flanks.

The device 10 is used for infiltration, delivery of ultrasonic energy, and aspiration of the surgical site. Device 10 includes cannula 100 which serves to provide infiltration, conduct ultrasonic energy from the ultrasound generating handpiece 200, and also provide a conduit for aspiration to the fluid system 400 used for infiltration and collection. Cannula 100 includes a channel 102 that provides the fluid path to fluid system 400 that includes components for both delivery of infiltration fluid to a surgical location and collection of lipoasiprate from the surgical location.

Referring again to FIG. 1, cannula 100 includes holes 105 near the end to allow passage of infiltration liquid to a surgical site and the aspiration of cellular material from the surgical site into channel 102 and eventually to system 400.

As shown in FIG. 1, handpiece 200 includes an ultrasonic driver assembly 202 that creates the ultrasonic vibrations that are delivered to the surgical site. More specifically, the ultrasonic driver assembly 202 includes a piezoelectric stack 204 that vibrates at a particular frequency in response to a signal generated by an amplifier console 206. Systems that embody the present invention can have frequencies that vary between about 35 kHz to about 45 kHz. For example, in one embodiment the frequency will be between about 36 kHz to about 42 kHz.

The piezoelectric stack 204 may be in some embodiments rings, wherein the back end of cannula 100 fits within the stack of rings. The ultrasonic driver assembly 202 is acoustically coupled to cannula 100 so that the vibrations are conducted through cannula 100 which is placed in contact with a patient for delivering ultrasonic energy to a surgical site.

Fluid system 400 shown in FIG. 1 includes a reservoir 402 for storing infiltration fluid. Pump 404 is connected to reservoir 402 and is used to deliver the infiltration fluid stored in reservoir 402 into channel 102 and eventually through holes 105 and to the surgical site. In the embodiment shown in FIG. 1, pump 404 connects to a side port 106 in cannula 100. When pump 404 is activated, the infiltration fluid flows into channel 102 through port 106 and flows out of holes 105.

In embodiments, the infiltration fluid is delivered at rates between about 50 ml/min to about 200 ml/min. As can be appreciated, delivering the fluid more quickly reduces the amount of time it takes to perform the procedure. However, delivering the fluid too quickly may be uncomfortable to a patient who may be conscious during delivery of the fluid. In one embodiment, pump 404 is configured to deliver infiltration fluid at about 150 ml/min. The amount of fluid delivered to the surgical site may range from about 250 cc to about 500 cc depending on the predetermined ratio of infiltration fluid to lipoaspirate. As indicated above, one preferred end result is to obtain about 250 cc of lipoaspirate. Accordingly, the ratios of infiltration fluid to lipoaspirate may range from about 1:1 to about 2:1.

Fluid system 400 also includes a reservoir 406 for storing lipoaspirate removed from the surgical site after infiltration fluid has been delivered to the surgical site and after ultrasonic energy has been applied to the surgical site. The lipoaspirate includes a portion of the infiltration fluid delivered to the surgical site as well as adipose tissue that may include adipocytes, stem cells, and other fluids. Pump 408 is connected to reservoir 406 and is used to create suction that removes the lipoaspirate from the surgical site and to the reservoir 406. In the embodiment shown in FIG. 1, pump 408 connects to cannula 100 through a second side port 108 in cannula 100. The lipoaspirate at the surgical site flows into channel 102 through holes 105 and flows into reservoir 406. The lipoaspirate can then be subjected to further processing to isolate and/or concentrate the stem cells in the lipoaspirate for later injection into a patient. The vacuum applied by pump 408 during aspiration of the lipoaspirate may range from about 10 inHg to about 30 inHg, such as about 15 inHg, about 20 inHg, or about 25 inHg.

As those with skill in the art will appreciate, reservoirs 402 and 406 can be any suitable sterile container for storing fluids including plastic fluid bags, plastic bottles, or glass bottles. Pumps 404 and 408 can be any suitable pump for delivering fluids to, and removing fluids from, surgical sites. In one embodiment, pumps 404 and 408 are peristaltic pumps.

In embodiments, system 400 may also include a filter 412. The use of the filter can aid in filtering out larger adipocytes thereby creating a more useful lipoaspirate for use in generating stem cells. Any suitable filter can be used as filter 410 including one or more screens or other mechanisms that remove larger adipocytes and other material from the lipoaspirate. For example, the filter 412 may include a number of filters of different sizes that are used in sequence to remove larger tissues first. The filters used to filter the lipoaspirate may range from about 200 μm to about 800 μm. In one specific embodiment, the filters for filtering lipoaspirate range from about 400 μm to about 500 μm. After the larger material has been removed from the lipoaspirate it can be processed to obtain the ADSC's.

System 400 illustrated in FIG. 1 is merely one example of an infiltration/collection system that may be used with the present invention. In other embodiments, system 400 may include only a single pump that can be activated to both deliver fluid to a surgical site and remove lipoaspirate from the surgical site. In these embodiments, cannula 100 may include only a single side port. In other embodiments, pumps 404 and 408 may be connected to the back end of cannula 100 instead of a side port. Also, although specific components of system 400 are shown in FIG. 1, other embodiments may include more or fewer components. Suitable pumps for use in fluid systems, such as system 400, are manufactured by Watson Marlow, Wilmington, Mass.

FIG. 1A illustrates an embodiment of another fluid system 400A with a different design than that of system 400. System 400A includes only a single compressor 420 and a single reservoir 422. In addition, system 400A connects to the back of cannula 100A, which does not include any side ports. System 400A operates pneumatically to deliver fluid through cannula 100A to a surgical site and remove lipoaspirate from the surgical site. In operation, compressor 420 pressurizes reservoir 422 with a gas (e.g., air, oxygen, nitrogen, etc.) which forces infiltration fluid in reservoir 422 to flow into cannula 100A and out of holes 105A into a surgical site. After cannula 100A has applied ultrasonic energy to the surgical site, compressor 420 is activated to create a vacuum within reservoir 422 and within cannula 100A. Lipoaspirate at the surgical site flows into cannula 100A through holes 105A and flows into reservoir 422. The lipoaspirate can then be subjected to further processing to isolate and/or concentrate the stem cells in the lipoaspirate for later injection into a patient.

System 400 and 400A are merely two embodiments of a fluid system (for infiltration and aspiration) that may be used, and the present invention is not limited thereto. In other embodiments, a fluid system may combine aspects of both system 400 and 400A such as: use of one or more compressors, use of one or more peristaltic pumps, use of one or more side port(s), use of a back port, use of one or more reservoirs, etc. Embodiments may also combine different aspects of the overall systems shown in FIG. 1 and FIG. 1A. For example, an embodiment may use a cannula that does not have any side ports so that the flow path of infiltration fluid and lipoaspirate passes directly through a handpiece. This can be combined with any of the features of infiltration/collection system 400 or 400A.

FIGS. 1B and 1C illustrate a cannula 100B that may be separated from a handpiece 200B. Cannula 100B and handpiece 200B may be used in embodiments of device 10. As shown in FIG. 1B, the cannula 100B slides into handpiece 200B. Cannula 100B includes a stop 150 that engages handpiece 200B and controls the amount of cannula 100B that slides into handpiece 200B and consequently the location of handpiece 200B along cannula 100B. The stop 150 may include features for securing handpiece 200B to cannula 100B including one or more of threads, tabs, channels, and/or tapered features that engage one or more corresponding features on handpiece 200B. FIG. 1C illustrates the handpiece 200B engaged with stop 150.

The embodiment shown in FIGS. 1B and 1C may be used in embodiments where cannula 100B is designed to be disposable or reposable. If cannula 100B is designed to be reposable, after each use, cannula 100B is separated from handpiece 200B allowing both cannula 100B and handpiece 200B to be separately sterilized. In those embodiments in which the cannula 100B is disposable, the handpiece 200B may be sterilized after each use, with a new cannula 100B being used each time.

FIG. 1D illustrates an embodiment of a device that has some components that are disposable and others that are not. Shown in FIG. 1D is a kit 152 with various components that are designed to be disposable. The components include cannula 100C, tubing 154, reservoir 158, and reservoir 160. In this embodiment, reservoirs 158 and 160 may be plastic fluid bags or other disposable container. Reservoir 158 may include a predetermined amount of infiltration fluid for use in a procedure. The components of kit 152 are used in combination with reusable components, such as handpiece 200C and fluid delivery system 400C that includes pumps 404C and 408C. As those with skill in the art will appreciate, when the components of kit 152 are connected to the reusable components, a device similar to device 10 shown in FIG. 1A is created and operates as described above with respect to FIG. 1A.

The use of a kit as shown in FIG. 1D is an efficient way of creating a device that minimizes the number of components that must be sterilized on site and also allows the amount of infiltration fluid to be predetermined as part of the kit. As one example, the reusable components such as handpiece 200C and fluid delivery system 400C may be on site at a hospital, e.g. in an emergency room, or other healthcare provider location where procedures are performed. When a patient requires a procedure, a healthcare provider may open up the prepackaged kit and assemble the disposable components with the reusable components to create the device that is used once on the patient. Because the amount of infiltration fluid is predetermined, the healthcare provider does not have to consider how much fluid is going into a patient but rather simply operates the device to deliver the predetermined amount of infiltration fluid in reservoir 158 to a patient, applies ultrasonic energy to the patient for a recommended period of time, and then removes the lipoaspirate from the patient and into container 160. After the procedure is done, the disposable components can be thrown away and the reusable components sterilized.

FIG. 1D is merely one embodiment of using components in a kit 152 that are designed to be disposable. In other embodiments, kit 152 may include more components such as a filter for filtering lipoaspirate, reservoirs for holding material filtered out of the lipoaspirate, pumps used in a fluid system for infiltration/aspiration, and even an ultrasonic handpiece. In other embodiments, the kit 152 may contain less than the components shown in FIG. 1D. For example, the kit 152 may contain only one reservoir 158, which is used to store the infiltration fluid and also used to store the lipoaspirate.

Referring again to FIG. 1, device 10 includes guide 500 which is positioned substantially parallel to a central axis of cannula 100. The separation between the cannula 100 and the guide 500 is fixed at a distance D. This fixed distance corresponds to the depth below the skin surface that cannula 100 should be used. The guide 500 is connected to handpiece 200 through a connector 502. The connector may be any combination of brackets, fasteners, gears, wheels, knobs, etc. In some embodiments, distance D is preset and cannot be changed by a user of device 10. In other embodiments, connector 502 may include a means to adjust distance D within some predefined range for example by using an electric motor or other adjustable mechanism.

It should be understood that although FIG. 1 illustrates guide 500 with a relatively straight shaft that is substantially parallel to cannula 500, the present invention is not limited to this embodiment. In other embodiments, the guide 500 may be angled or include a shaft with some curvature. The shaft is also not limited to any particular cross sectional shape. These variations, and others, can still maintain a distance D between at least a portion of the guide 500 and cannula 100 to limit the depth of cannula 100 within a patient.

FIG. 2 illustrates how guide 500 is useful for controlling the depth of cannula 100. For simplicity purposes, FIG. 2 does not show some of the features, such as system 400, shown in FIG. 1. The cannula 100 is inserted into the patient's body via a small incision. As shown in FIG. 2 the patient's body may include layers of skin 602, a subcutaneous layer of fat 604 from which the adipose tissue and stem cells are removed, and some other anatomical layer 606 (e.g. muscle tissue). Although shown as distinct layers in the drawings, in fact, layers such as 604 and 606 can be interdispersed particularly at their boundary. One of the advantages of having a system “tuned” for harvesting adipose tissue is that it should minimize fragmentation and aspiration on non-fat tissues should the cannula 100 come into contact with non-fat tissues during operation. The cannula 100 is generally moved parallel to the skin's top surface 600, using guide 500 as a depth gauge. Guide 500 is positioned on the top surface 600 of the skin 602, as shown in FIG. 2.

FIG. 2A includes arrows that illustrate the movement of cannula 100 and guide 500. As shown in FIG. 2A, cannula 100 is inserted into a patient. A user may manipulate skin 602, such as by pressing down on a patient, to allow cannula 100 to move substantially parallel to a portion of the skin 602 as indicated by arrow 610. As can be appreciated, guide 500 slides across the top surface of skin 602 when cannula 100 is moved back and forth as shown by arrows 610. Guide 500 limits the depth that cannula 100 can reach within a patient. If a user attempts to move the tip of cannula 100 deeper into a patient, i.e., in the direction indicated by arrow 612, guide 500, which is against the top surface of skin 602, will prevent the tip of cannula 100 from moving very far into the patient. This feature prevents the user, which may not be as skilled as a surgeon, from damaging the anatomical layer 606 of the patient.

In embodiments, the distance between at least a portion of the guide 500 and the cannula 100 may be more than about 1 cm, and less than about 3 cm, such as about 1.5 cm, about 2.0 cm, or about 2.5 cm. This would help to assure that the cannula 100 is neither too shallow nor too deep. As can be seen in FIG. 2, if the cannula 100 is too deep it can damage anatomical layer 606. If however, cannula 100 is too shallow it will not be within the fat layer from which the adipose tissue and stem cells are removed. Once suitably positioned within a fat bearing region, the tissue is infiltrated through the cannula with a fixed amount of infiltration fluid. The amount can be fixed because it is a defined ratio of the total aspirate to be removed (typically in a 1:1 to 2:1 ratio). The tissue can also be imaged using standard ultrasound imaging techniques, alternately with an ultrasound probe head 700 attached to guide 500, as shown in FIG. 3.

In one embodiment, the probe head 700 is used to set the distance between guide 500 and cannula 100. A user can use the probe head 700 to scan the surgical site and determine the depth to the muscle layer. The distance between the guide 500 and the cannula 100 can then be set by the user to prevent the cannula 100 from puncturing the muscle layer. In other embodiments, the use of ultrasound to locate the muscle layer and set the distance between the guide 500 and the cannula 100 may be performed using a separate ultrasound imaging system instead of the probe head 700.

As noted above, guide 500 is designed to prevent cannula 100 from being too shallow or too deep within a patient. The length of guide 500 will therefore depend on the length of cannula 100. In embodiments, cannula 100, and therefore guide 500, is designed to have a length that is optimal in removing adipose tissue from a surgical location on a patient. As those with skill in the art will appreciate, different locations in a human body have different characteristics that may be considered when designing cannula 100 and guide 500. For example, some locations may have relatively thick layers of fat while others have thinner areas. These characteristics can be considered for example to optimize the features (e.g., length, width, construction material) of cannula 100 and guide 500 for removal of tissue from specific locations in a patient.

FIG. 9 illustrates an alternative design showing a stop 902 that may be used in some devices embodying the present invention. As illustrated in FIG. 9, stop 902 is used to limit the depth of cannula 100 in a patient. As can be appreciated, stop 900 and guide 500 are merely examples of devices that provide for limiting the depth to which a cannula can be inserted into a patient. Other designs that provide the same functionality are within the scope of the present invention.

Referring again to operation of the device 10, once the tissue is infiltrated through cannula 100 with tumescent fluid, which may contain a number of different components such as saline and anesthetic, the ultrasound handpiece 200 is energized, causing ultrasonic vibrations at the end of the cannula 100. As can be appreciated, the infiltration fluid may include some anesthetic that requires some time to properly numb the surgical site. As a result, in some embodiments, the ultrasonic driver assembly within handpiece 200 is programmed to be inoperable for a preset period of time, e.g., about 15 minutes, after the delivery of the infiltration fluid. This programming provides a level of safety that ensures that the user cannot prematurely apply the ultrasonic energy before the anesthetic has time to take effect.

When the ultrasonic energy is applied, the vibrations act to selectively dislodge the fat at the surgical site, through means described in literature and patents and otherwise known to one of ordinary skill in the art. Embodiments of the present invention operate at frequencies that are preset, which eliminates the need of a user to select a frequency. The frequencies can range from about 35 kHz to about 45 kHz. In some embodiments, the frequency may be from about 36 kHz to about 42 kHz or alternatively from about 36 kHz to about 40 kHz. In one embodiment, the frequency may be from about 36 kHz to about 38 kHz. In embodiments, the system operates at frequencies typical of the VASER® system manufactured by Sound Surgical Technologies of Louisville, Colo. These systems in embodiments operate at frequencies at or about 36 kHz. Once the fat cells have been put into suspension, the same cannula 100 is used to withdraw the fat out of the body and into a collection system, e.g., 400 (FIG. 1) or 400A (FIG. 1A), for further processing.

The device 10 may be configured in some embodiments to allow a second infiltration step, followed by subsequent ultrasonic energy application and aspiration steps. These second series of steps may be allowed if the amount of lipoaspirate initially obtained is below the amount necessary to process into ADSC's, e.g., less than 250 cc.

The ultrasonic vibration and aspiration process can be controlled so that it is done simultaneously if desired. In contrast to other systems which may do this simultaneous action automatically, the present system can in embodiments be controlled to only energize the handpiece for a fixed time and amplitude, corresponding to the amount of infiltration fluid introduced into the patient. The predetermined time and the predetermined amplitude of the applied ultrasonic energy may be based on the amount of infiltration fluid and/or the anticipated amount of lipoaspirate to be removed from the patient. Applying a fixed amount of ultrasonic energy provides a level of safety that is not found in other systems. Devices embodying the present invention can be configured to provide ultrasonic energy with amplitudes ranging from about 30 μm to about 80 μm. More specifically, the ultrasonic energy may have amplitudes from about 50 μm to about 60 μm. The ultrasonic energy may be applied for about 2 to about 4 minutes, such as about 2.5 minutes, about 3 minutes, or about 3.5 minutes.

The cannula 100 can be made such that it passes through the entire length of the ultrasound handpiece with the connection to the rest of the system (infiltration, aspiration) at the end opposite that put into the patient (see FIG. 2). As those skilled in the art will realize, the places at which the connection is made to the cannula must be controlled with regard to the standing wave locations in the cannula.

If the cannula passes through the length of the handpiece, the connection point of the ultrasonic energy must be controlled, again, as would be understood by those skilled in the art. Since the cannula is used for all three portions of the procedure, it must be designed to accommodate all the various requirements. In other words, the holes at the end of the cannula must be designed taking into account the ultrasound vibrations. In order to be used safely for infiltration, the tip of the cannula must be closed (blunt), which again, must be incorporated into the ultrasound design problem, as stress concentrations can occur near the tip.

As one example, FIG. 4 shows a wave 480 that illustrates the vibrational energy transmitted through a cannula 100B. Wave 480 includes nodes 482 and 484 and antinodes 486 and 488. The nodes are where the longitudinal ultrasonic vibration has maximum amplitude and the antinodes are where the longitudinal ultrasonic vibration has minimal amplitude. Holes 490 and 492 correspond to nodal locations, which are locations that undergo the least amount of stress and therefore are less likely to fail.

Shown in FIGS. 5A-7A are cannula tips that may be used in embodiments of the present invention. FIGS. 5A, 6A, and 7A illustrate cross sectional views of cannulas taken parallel to a center axis of a cannula. FIGS. 5B, 6B, and 7B illustrate cross sectional views of cannulas taken perpendicular to a center axis of a cannula. FIGS. 5A-7A are not drawn to scale and are used merely to illustrate different cannula designs contemplated by the present invention. As part of limiting the amount of trauma that may be caused to a patient, cannulas used with embodiments of the present invention may be relatively short in length compared with cannulas used in cosmetic procedures. As noted above, devices embodying the present invention will be operated by users that are not as trained as surgeons. Accordingly, embodiments may utilize cannulas that cannot be inserted very far into a patient. As one example, the cannulas may be from about 8 cm to about 12 cm in length depending on the location of the surgical site. The length of the cannula will depend on the frequency of the ultrasonic energy conducted by the cannula. The length of the cannula will be a fixed multiple of the wavelength of the frequency used, which as noted above may be from about 36 kHz to about 42 kHz in some embodiments.

FIGS. 5A and 5B are views of a cannula similar to the cannula 100 (FIG. 1). The cannula of FIGS. 5A and 5B however does not include a rounded tip with holes. Rather, the channel 550, which provides a pathway for infiltration fluid and lipoaspirate, extends through the front end of the cannula. In embodiments, this allows a larger amount of infiltration fluid and lipoaspirate to flow through channel 550. In embodiments, the cannula illustrated in FIGS. 5A and 5B may have an outer diameter of about 3.5 mm to about 4.5 mm, such as about 4 mm.

FIGS. 6A and 6B are views of a cannula that includes a probe 662 and an outer sheath 660. In some embodiments, probe 662 is used to transmit ultrasonic energy, and sheath 660 does not transmit ultrasonic energy. As can be seen in FIGS. 6A and 6B, a channel 664 is formed between an inner surface of sheath 620 and an outer surface of probe 662. Channel 664 is used to deliver infiltration fluid to a surgical site and remove lipoaspirate from the surgical site. One feature of the cannula shown in FIGS. 6A and 6B is that the probe 662 can be used to apply ultrasonic energy to the surgical site during infiltration, after infiltration, and during aspiration. The vibration of probe 662 during aspiration will assist in breaking up groups of adipocytes that stick together. The end result is a lipoaspirate that may be more easily processed for removing stem cells. The inner probe can have an outer diameter that ranges from about 2.2 mm to about 2.9 mm, with the outer sheath having an outer diameter ranging from about 3.5 mm to about 4.5 mm.

In other embodiments, probe 662 does not extend beyond sheath 660. In these embodiments, sheath 660 is used to transmit ultrasonic energy to the surgical site. In addition, probe 662 can be vibrated during aspiration to break up groups of adipocytes in the lipoaspirate as it travels through channel 664.

FIGS. 7A and 7B are views of a cannula that is similar to the cannula illustrated by FIGS. 6A and 6B. The cannula of FIGS. 7A and 7B includes a probe 762 and an outer sheath 760, which create a channel 764. As shown in FIGS. 7A and 7B the probe 762 does not extend beyond sheath 760. Thus, a ring 770 is included at the end of the cannula to vibrate the end of the cannula and apply ultrasonic energy to a surgical site. In embodiments, the probe 762 also vibrates and can be vibrated during aspiration. The vibration of probe 762 during aspiration will assist in breaking up groups of adipocytes that stick together. As with the cannula shown in FIGS. 6A and 6B, the end result is a lipoaspirate that may be more easily processed for removing stem cells.

As can be appreciated, the cannula designs shown in FIGS. 5A-7B are merely some examples of cannulas that may be used with embodiments of the present invention. Other designs can be used for delivery of infiltration fluid to a surgical site, application of ultrasonic energy to the surgical site, and removal of aspirate from the surgical site.

FIG. 8 shows a block diagram of a computing device 800. The computing device 800 can be used in embodiments to control various features of the embodiments of the present invention described above. Computing device 800 includes a processor 802 and memory 804. The processor and the memory may be connected using one or more buses. Processor 802 can be configured to execute instructions embodied in software that may be stored in memory 804. Memory 804 also stores data used by processor 802. In embodiments, memory 804 may store software that when accessed and executed by processor 802 causes the processor to: control the activation and deactivation of pumps or compressors to deliver infiltration fluid to a surgical site or remove lipoaspirate from the surgical site. The processor may also execute software that controls the ultrasonic energy delivered to a surgical site (amplitude and duration) and the ultrasound used to image the surgical site being treated. As part of this control, processor 802 and memory 804 may receive store, manipulate, and calculate data. In other embodiments, the control of various features may be implemented using hardware logic 806, instead of, or in addition to, software instructions stored in memory 804 executed by processor 802.

### EXAMPLE

A device that includes a cannula is used to remove lipoaspirate from a surgical location in a patient. The device also includes a guide that prevents the cannula from being inserted too deeply into the patient. The guide includes a shaft that extends substantially parallel to the cannula. The distance between the guide and the cannula is set to about 2.5 cm.

The cannula is in fluid communication with a fluid delivery system that is used to deliver 300 cc of an infiltration fluid through the cannula to the surgical location in a patient at a rate of 150 ml/min. The infiltration fluid includes saline as well as other components such as a local anesthetic (e.g., lidocaine) and a vascular constrictor (e.g. epinephrine). The fluid system includes a pump that delivers the fluid at the rate of 150 ml/min.

The cannula is acoustically connected to an ultrasonic driver assembly that is preset to a frequency of 36 kHz to apply ultrasonic energy with amplitude of 55 μm for 3 minutes to the surgical location. The cannula conducts the ultrasonic energy from the driver assembly to its tip where it is applied to the surgical location.

After the ultrasonic energy is applied to the surgical location, the fluid system is used to remove lipoaspirate from the patient through the cannula by applying a vacuum. The fluid system applies a vacuum of 15 inHg for removing the lipoaspirate from the surgical site. 250 cc of lipoaspirate are removed from the surgical location.

As those with skill in the art will appreciate, different locations in a human body have different characteristics that may be considered when designing aspects of embodiments of the present invention for use in harvesting adipose cells from a patient. As noted above, some surgical locations in a patient may have relatively thick layers of fat while others have thinner areas. These characteristics can be considered for example to optimize various features of embodiments of the present invention (some of which are described herein and illustrated in the drawings). These features include but are not limited to: length, width, and spacing of the cannula and the guide; amount of infiltration fluid used; composition of infiltration fluid; the amount of pressure or vacuum used in infiltration or aspiration steps; and the amount of ultrasound delivered to the surgical location. These features, and others, can be modified to optimize the harvesting of adipose tissue from different surgical locations in a patient.

As will be appreciated by those of skill in the art, the embodiments described herein are distinct from conventional ultrasonic assisted lipoplasty. Among other things, the apparatus and method of the present invention differ from the ultrasonic assisted lipoplasty equipment and procedures commonly employed in that: (1) a cannula is used for infiltration, ultrasound exposure, and aspiration; (2) a fixed amount of infiltration fluid is injected into the surgical site in specific ratio with the amount of lipoaspirate to be removed; (3) a fixed amount of ultrasound energy, both in amplitude and time, is delivered to the surgical site commensurate with the amount of infiltration and aspiration; (4) an ultrasonic cannula is employed with holes positioned to not interfere with the ultrasonic action; and (5) the device includes a guide that limits the depth to which the cannula can be inserted into a patient. The features can be combined in various embodiments to provide safe and efficient systems and methods for removing adipose tissue from a patient by medical personnel that are less skilled than a typical cosmetic surgeon.

As a result, the apparatus and method of the present invention provide a simple device and technique that can be operated efficiently and successfully by non-cosmetic surgeons, for the purpose of harvesting ADSC's and processing for regenerative medicine. A disposable cannula can be used to improve sterility and reduce processing costs.

Reference has been made throughout this specification to “embodiment,” “one embodiment,” “an embodiment,” “another embodiment,” and “some embodiment” meaning that a particular described feature, structure, or characteristic is included in at least one embodiment of the present invention. Thus, usage of such phrases may refer to more than just one embodiment. Furthermore, the described features, structures, or characteristics may be combined in any suitable manner in one or more embodiments.

One skilled in the relevant art may recognize, however, that the invention may be practiced without one or more of the specific details, or with other methods, resources, materials, etc. In other instances, well known structures, resources, or operations have not been shown or described in detail merely to avoid obscuring aspects of the invention.

While example embodiments and applications of the present invention have been illustrated and described, it is to be understood that the invention is not limited to the precise configuration and resources described above. Various modifications, changes, and variations apparent to those skilled in the art may be made in the arrangement, operation, and details of the methods and systems of the present invention disclosed herein without departing from the scope of the claimed invention.

I claim:

1. A method of obtaining tissue utilizing a device comprising a cannula, the method comprising:
limiting the depth of the cannula using a guide positioned at a distance from the cannula;
delivering through a channel of the cannula a fixed amount of infiltration fluid in specific ratio with an amount of lipoaspirate to be removed, wherein the infiltration fluid is delivered through at least one hole in the cannula;
applying a fixed amount of ultrasonic energy using the distal end of the cannula, wherein the ultrasonic energy is applied at a predetermined amplitude and for a predetermined period of time; and
removing the lipoaspirate through the channel of the cannula.

2. The method of claim 1, wherein the predetermined amplitude and the predetermined period of time are based on the amount of infiltration fluid delivered and the amount of lipoaspirate to be removed.

3. The method of claim 1, wherein the ultrasonic energy is conducted through at least a portion of the cannula to the distal end of the cannula.

4. The method of claim 3, wherein ultrasonic energy is conducted through at least a portion of the cannula to the distal end of the cannula during the removing of the lipoaspirate.

5. The method of claim 3, wherein the at least one hole is positioned at a location in the cannula that is affected by lower amounts of stress when conducting the ultrasonic energy.

6. The method of claim 3, wherein the plurality of holes are positioned at locations in the cannula that are affected by stress when conducting the ultrasonic energy.

7. The method of claim 1, further comprising applying ultrasonic energy to create an image of the location from which the lipoaspirate will be removed.

8. The method of claim 1, wherein the fixed amount of infiltration fluid is delivered from a first container and the lipoaspirate is stored within the first container.

9. The method of claim 1, further comprising filtering the lipoaspirate after the removing the lipoaspirate.

10. The method of claim 1, further comprising processing the lipoaspirate to generate adipose derived stem cells.

11. A device for harvesting adipose tissue from a medical patient, the device comprising:
a cannula for delivering infiltration fluid to a surgical site in a patient and removing lipoaspirate from the surgical site, wherein the distal end of the cannula is configured to be positioned within subcutaneous tissue of the patient;
an ultrasonic driver assembly acoustically coupled to a portion of the cannula and configured to generate ultrasonic energy that is transmitted to the distal end of the cannula and to the subcutaneous tissue of the patient, wherein the ultrasonic driver assembly is configured to deliver a fixed amount of ultrasonic energy to the subcutaneous tissue by applying the ultrasonic energy at a predetermined amplitude for a predetermined period of time; and
a guide positioned at a predetermined distance from the cannula to limit the depth of the distal end of the cannula within the patient.

12. The device of claim 11, wherein the ultrasonic driver assembly is part of a handpiece and the guide is connected to the handpiece.

13. The device of claim 11, wherein the cannula is configured to slide into the handpiece and comprises a stop that engages with the handpiece.

14. The device of claim 11, wherein the guide comprises a shaft that is substantially parallel to a central axis of the cannula.

15. The device of claim 11, wherein the predetermined distance can be changed within a predetermined range.

16. A device for harvesting adipose tissue from a medical patient, the device comprising:
a cannula comprising:
a channel,
at least one port, and
at least one hole on a distal end of the cannula, wherein the distal end of the cannula is designed to be positioned within subcutaneous tissue of a patient;
a fluid system connected to the at least one port of the cannula and used to deliver a fixed amount of infiltration fluid into the channel of the cannula and to the subcutaneous tissue of the patient through the at least one hole, wherein the fluid system is also used to remove lipoaspirate from the subcutaneous tissue of the patient through the at least one hole and the central channel of the cannula;
an ultrasonic driver assembly acoustically coupled to at least a portion of the cannula and configured to generate ultrasonic energy that is transmitted to the distal end of the cannula and to the subcutaneous tissue of the patient; and
a guide positioned at a predetermined distance from the cannula, wherein the guide limits the depth of the distal end of the cannula within the patient.

17. The device of claim 16, wherein the at least one port is located at a proximate end of the cannula.

18. The device of claim 16, wherein the at least one port is located between a proximate end of the cannula and a distal end of the cannula.

19. The device of claim 16, wherein the fluid system further comprises:
a first container for storing the fixed amount of infiltration fluid;
a first pump for delivering the fixed amount of infiltration fluid from the first container to the surgical site;
a second pump for removing the lipoaspirate from the surgical site; and
a second container for storing the lipoaspirate.

20. The device of claim 16, wherein the fluid system further comprises:
a container for storing the fixed amount of infiltration fluid and the lipoaspirate;
a pump for delivering the fixed amount of infiltration fluid from the container to the surgical site and for removing the lipoaspirate from the surgical site to the container.
